(19) United States
(12) Patent Application Publication
(10) Pub. No.: US 2007/0299522 A1
(43) Pub. Date: Dec. 27, 2007

(54) Orthopedic implant with integrated bone screw

(76) Inventor: Carlos E. Gil, Collierville, TN (US)

Correspondence Address: LARSON NEWMAN ABEL POLANSKY & WHITE, LLP, 5914 WEST COURTYARD DRIVE, SUITE 200, AUSTIN, TX, 78730, US

(21) Appl. No.: 11/449,545

(22) Filed: Jun. 8, 2006

(51) Int. Cl. A61F 2/44

(52) U.S. Cl. 623/17.11

(57) ABSTRACT

An implant is disclosed and can include a component. The implant can further include a bone screw having a head portion that can be integrated within the component. The bone screw can be held in place with respect to the component by a screw engagement hole that only partially surrounds the head portion of the bone screw.

### FIELD OF THE DISCLOSURE

The present disclosure relates generally to orthopedics and orthopedic surgery. More specifically, the present disclosure relates to orthopedic implants.

### BACKGROUND

A human body contains over two hundred bones that, for various reasons, can require orthopedic intervention. Furthermore, the human body contains many synovial joints that can fail. In certain circumstances, diseased or damaged joints can be totally or partially replaced with prosthetic implants. One of the primary governing aspects of long-term success of an orthopedic implant is successful attachment to surrounding bone structure. An application that can be especially challenging due to the range of motion and loading involved is spinal arthroplasty.

In human anatomy, the spine is a generally flexible column that can take tensile and compressive loads. The spine also allows bending motion and provides a place of attachment for keels, muscles and ligaments. Generally, the spine is divided into three sections: the cervical spine, the thoracic spine and the lumbar spine. The sections of the spine are made up of individual bones called vertebrae. Also, the vertebrae are separated by intervertebral discs, which are situated between adjacent vertebrae.

The intervertebral discs function as shock absorbers and as joints. Further, the intervertebral discs can absorb the compressive and tensile loads to which the spinal column may be subjected. At the same time, the intervertebral discs can allow adjacent vertebral bodies to move relative to each other a limited amount, particularly during bending, or flexure, of the spine. Thus, the intervertebral discs are under constant muscular and/or gravitational pressure and generally, the intervertebral discs are the first parts of the lumbar spine to show signs of deterioration.

Facet joint degeneration is also common because the facet joints are in almost constant motion with the spine. In fact, facet joint degeneration and disc degeneration frequently occur together. Generally, although one may be the primary problem while the other is a secondary problem resulting from the altered mechanics of the spine, by the time surgical options are considered, both facet joint degeneration and disc degeneration typically have occurred. For example, the altered mechanics of the facet joints and/or intervertebral disc may cause spinal stenosis, degenerative spondylolisthesis, and degenerative scoliosis.

### BRIEF DESCRIPTION OF THE DRAWINGS

FIG. 1 is a lateral view of a portion of a vertebral column;

FIG. 2 is a lateral view of a pair of adjacent vertrebrae;

FIG. 3 is a top plan view of a vertebra;

FIG. 4 is a perspective view of an intervertebral prosthetic disc;

FIG. 5 is a lateral plan view of the intervertebral prosthetic disc;

FIG. 6 is an anterior plan view of the intervertebral prosthetic disc;

FIG. 7 is an inferior plan view of the intervertebral prosthetic disc;

FIG. 8 is an exploded perspective view of the intervertebral prosthetic disc;

FIG. 9 is a lateral plan view of a superior component of the intervertebral prosthetic disc;

FIG. 10 is an anterior plan view of the superior component of the intervertebral prosthetic disc;

FIG. 11 is a perspective view of the superior component of the intervertebral prosthetic disc;

FIG. 12 is another anterior plan view of the superior component of the intervertebral prosthetic disc;

FIG. 13 is a perspective view of an inferior component of the intervertebral prosthetic disc;

FIG. 14 is a lateral plan view of the inferior component of the intervertebral prosthetic disc;

FIG. 15 is another lateral plan view of the inferior component of the intervertebral prosthetic disc;

FIG. 16 is an anterior plan view of the inferior component of the intervertebral prosthetic disc; and

FIG. 17 is an exploded lateral view of the intervertebral prosthetic disc installed within an intervertebral space between a pair of adjacent vertrebrae.

### DETAILED DESCRIPTION OF THE DRAWINGS

An intervertebral prosthetic disc is disclosed and can be installed within an intervertebral space between a superior vertebra and an inferior vertebra. The intervertebral prosthetic disc can include an inferior component and a superior component that can be movably engaged with the inferior component. The superior component can include a superior bracket. Further, a screw engagement hole can be established in the superior bracket. The screw engagement hole can include a semi-cylindrical hole having an inner surface.

In another embodiment, an intervertebral prosthetic disc is disclosed and can be installed within an intervertebral space between a superior vertebra and an inferior vertebra. The intervertebral prosthetic disc can include an inferior component that can have a depression formed therein and a superior component that can have a projection that can movably engage the depression. A bone screw having a head portion can be integrated within the superior component. Moreover, the bone screw can be held in place by a screw engagement hole that only partially surrounds the head portion of the bone screw.

In yet another embodiment, an implant is disclosed and can include a component. The implant can further include a bone screw having a head portion that can be integrated within the component. The bone screw can be held in place with respect to the component by a screw engagement hole that only partially surrounds the head portion of the bone screw.

### Description of Relevant Anatomy

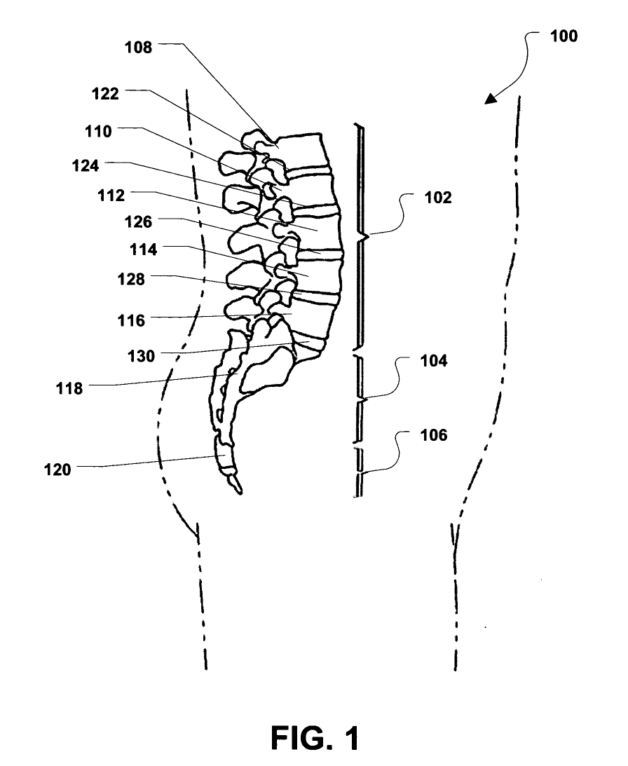

Referring initially to FIG. 1, a portion of a vertebral column, designated 100, is shown. As depicted, the vertebral column 100 includes a lumbar region 102, a sacral region 104, and a coccygeal region 106. As is known in the art, the vertebral column 100 also includes a cervical region and a thoracic region. For clarity and ease of discussion, the cervical region and the thoracic region are not illustrated.

As shown in FIG. 1, the lumbar region 102 includes a first lumbar vertebra 108, a second lumbar vertebra 110, a third lumbar vertebra 112, a fourth lumbar vertebra 114, and a fifth lumbar vertebra 116. The sacral region 104 includes a sacrum 118. Further, the coccygeal region 106 includes a coccyx 120.

As depicted in FIG. 1, a first intervertebral lumbar disc 122 is disposed between the first lumbar vertebra 108 and the second lumbar vertebra 110. A second intervertebral lumbar disc 124 is disposed between the second lumbar vertebra 110 and the third lumbar vertebra 112. A third intervertebral lumbar disc 126 is disposed between the third lumbar vertebra 112 and the fourth lumbar vertebra 114. Further, a fourth intervertebral lumbar disc 128 is disposed between the fourth lumbar vertebra 114 and the fifth lumbar vertebra 116. Additionally, a fifth intervertebral lumbar disc 130 is disposed between the fifth lumbar vertebra 116 and the sacrum 118.

In a particular embodiment, if one of the intervertebral lumbar discs 122, 124, 126, 128, 130 is diseased, degenerated, damaged, or otherwise in need of repair, treatment of that intervertebral lumbar disc 122, 124, 126, 128, 130 can be effected in accordance with one or more of the embodiments described herein.

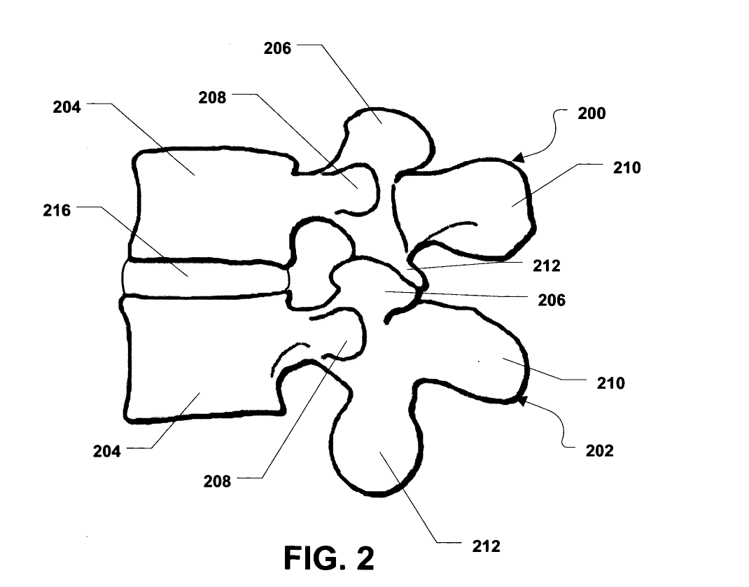

FIG. 2 depicts a detailed lateral view of two adjacent vertebrae, e.g., two of the lumbar vertebra 108, 110, 112, 114, 116 shown in FIG. 1. FIG. 2 illustrates a superior vertebra 200 and an inferior vertebra 202. As shown, each vertebra 200, 202 includes a vertebral body 204, a superior articular process 206, a transverse process 208, a spinous process 210 and an inferior articular process 212. FIG. 2 further depicts an intervertebral disc 216 between the superior vertebra 200 and the inferior vertebra 202.

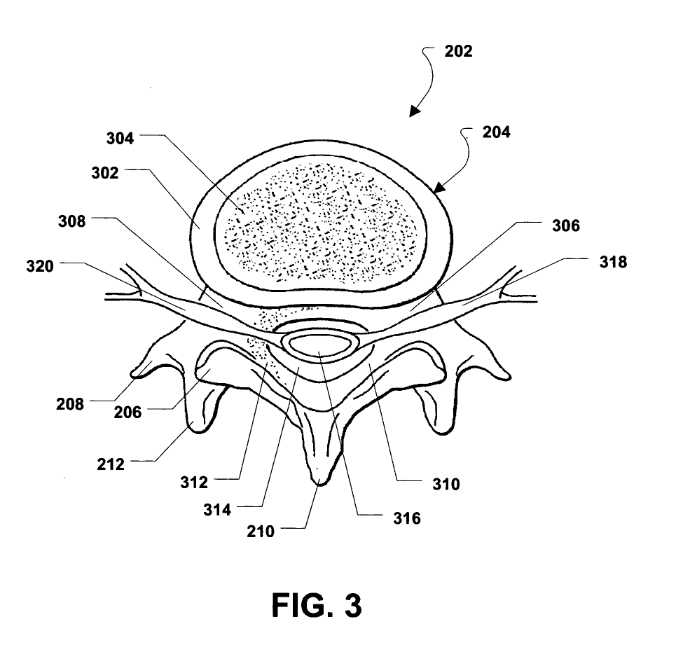

Referring to FIG. 3, a vertebra, e.g., the inferior vertebra 202 (FIG. 2), is illustrated. As shown, the vertebral body 204 of the inferior vertebra 202 includes a cortical rim 302 composed of cortical bone. Also, the vertebral body 204 includes cancellous bone 304 within the cortical rim 302. The cortical rim 302 is often referred to as the apophyseal rim or apophyseal ring. Further, the cancellous bone 304 is softer than the cortical bone of the cortical rim 302.

As illustrated in FIG. 3, the inferior vertebra 202 further includes a first pedicle 306, a second pedicle 308, a first lamina 310, and a second lamina 312. Further, a vertebral foramen 314 is established within the inferior vertebra 202. A spinal cord 316 passes through the vertebral foramen 314. Moreover, a first nerve root 318 and a second nerve root 320 extend from the spinal cord 316.

It is well known in the art that the vertebrae that make up the vertebral column have slightly different appearances as they range from the cervical region to the lumbar region of the vertebral column. However, all of the vertebrae, except the first and second cervical vertebrae, have the same basic structures, e.g., those structures described above in conjunction with FIG. 2 and FIG. 3. The first and second cervical vertebrae are structurally different than the rest of the vertebrae in order to support a skull.

### Description of an Intervertebral Prosthetic Disc

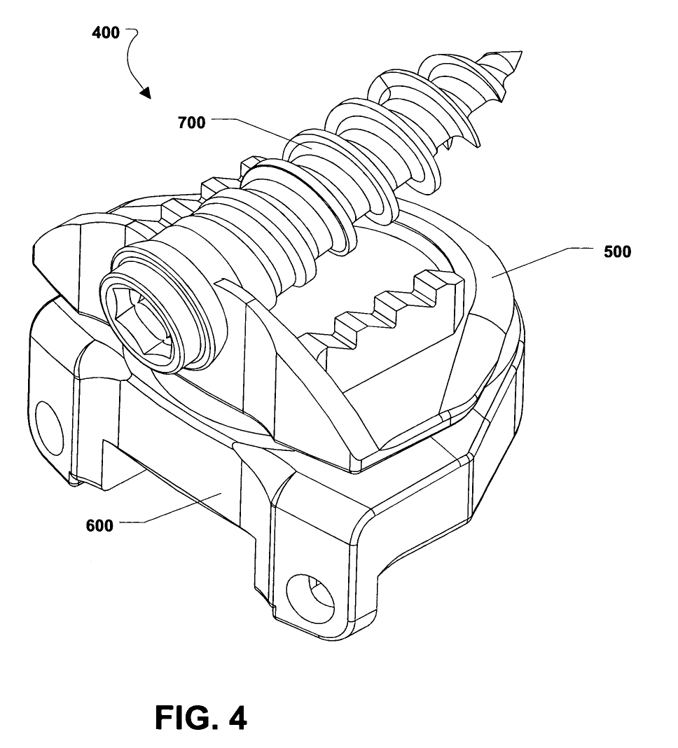

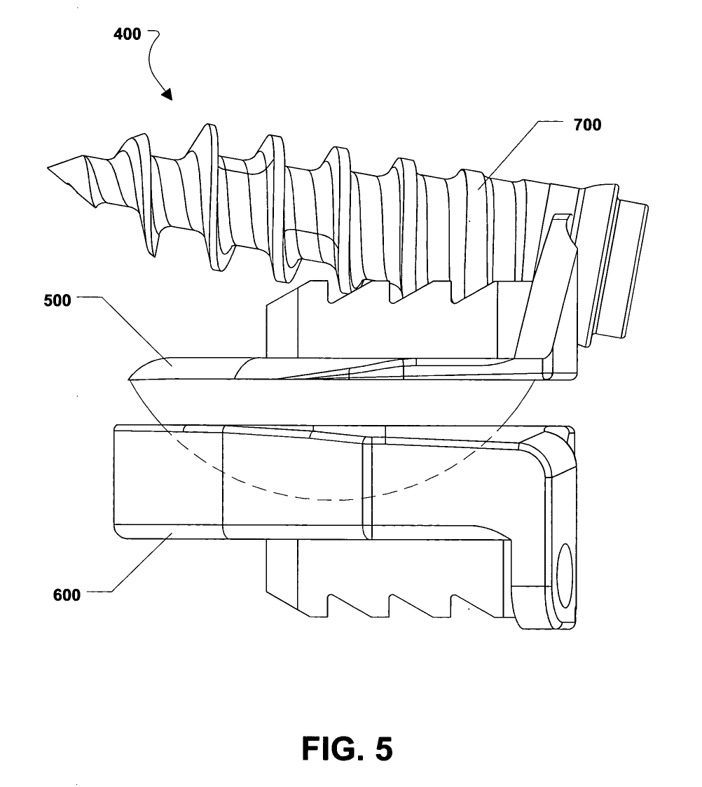

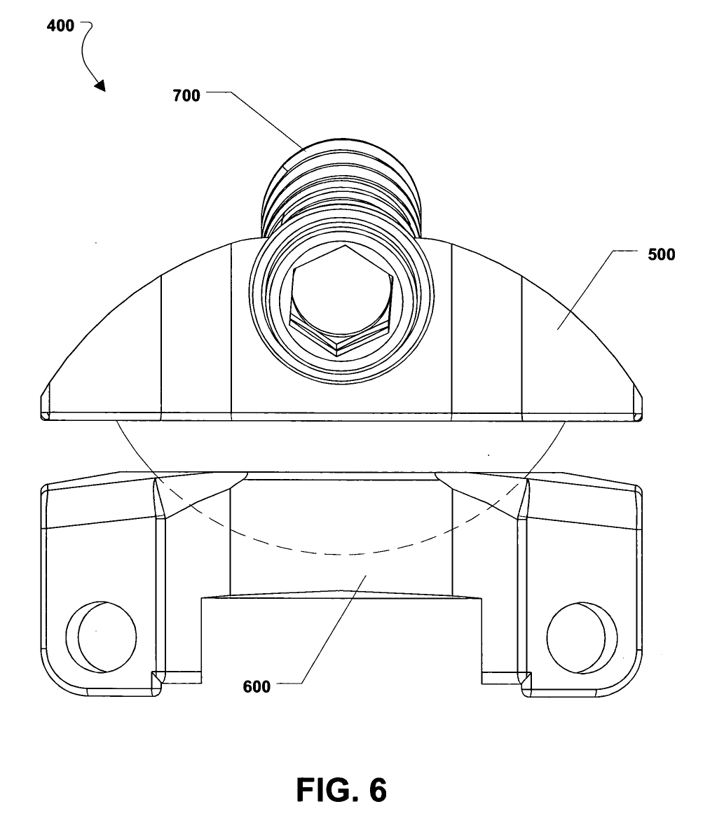

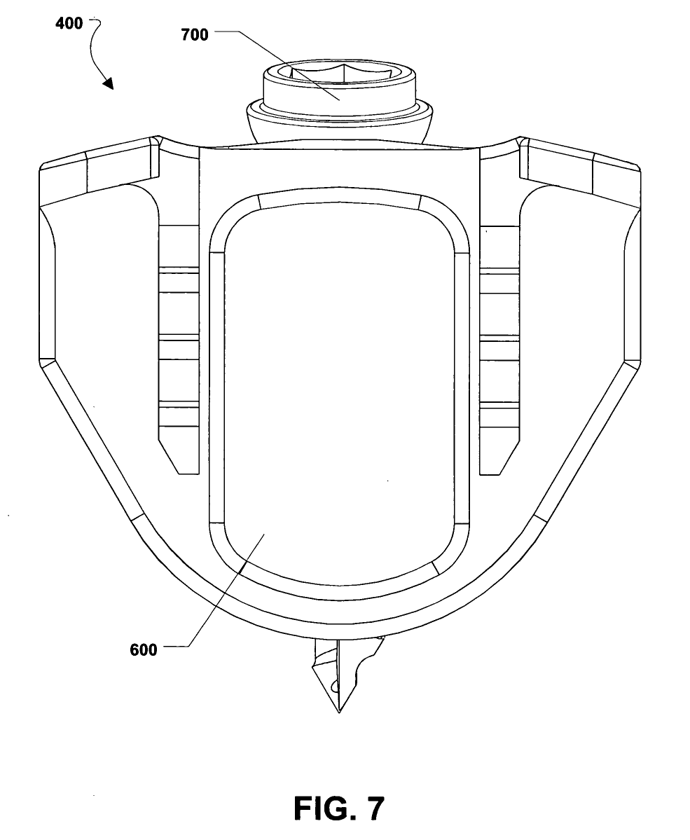

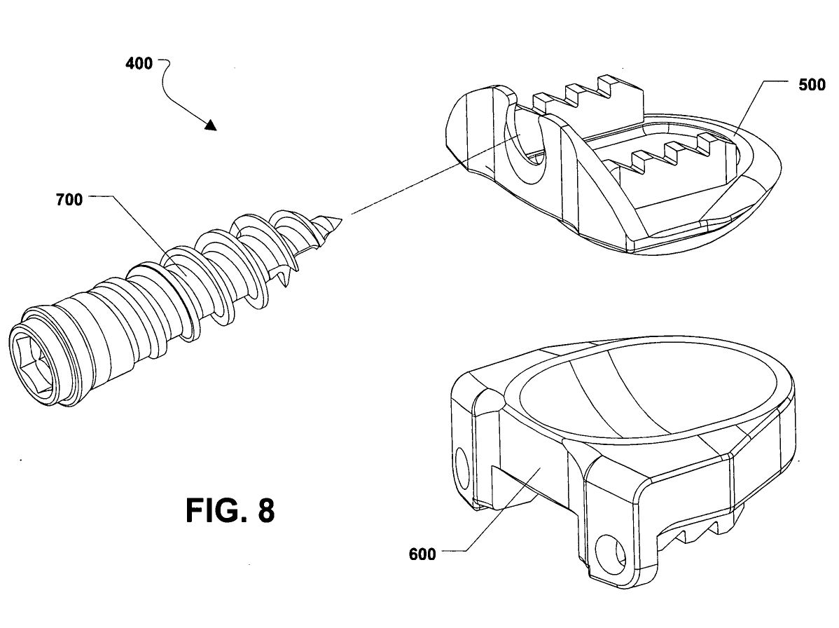

Referring to FIGS. 4 through 8 an intervertebral prosthetic disc is shown and is generally designated 400. As illustrated, the intervertebral prosthetic disc 400 includes a superior component 500 and an inferior component 600. Moreover, the intervertebral prosthetic disc 400 includes a bone screw 700 that can be engaged with the superior component. In a particular embodiment, the components 500, 600 and the bone screw 700 can be made from one or more extended use approved medical materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyaryletherketone (PAEK) materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyetherketoneetherketoneketone (PEKEKK), or a combination thereof. Alternatively, the components 500, 600 and the bone screw 700 can be made from any other substantially rigid biocompatible materials.

### Description of the Superior Component

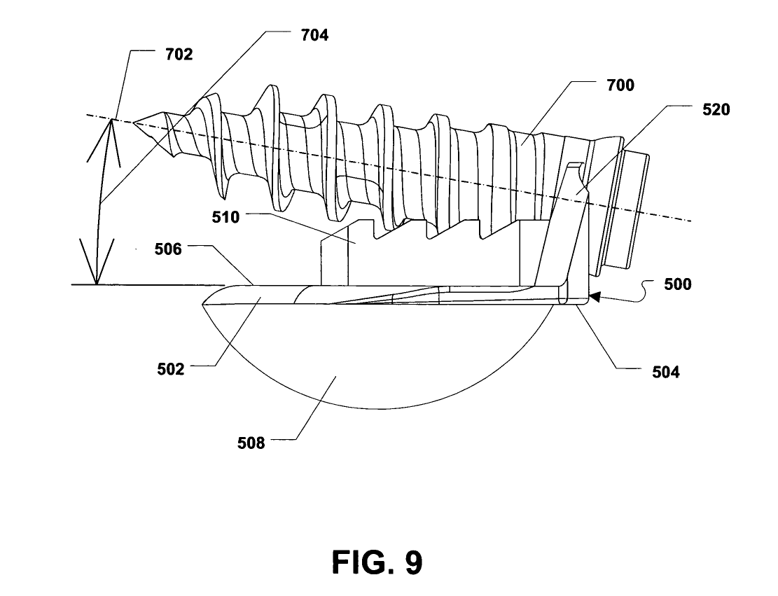

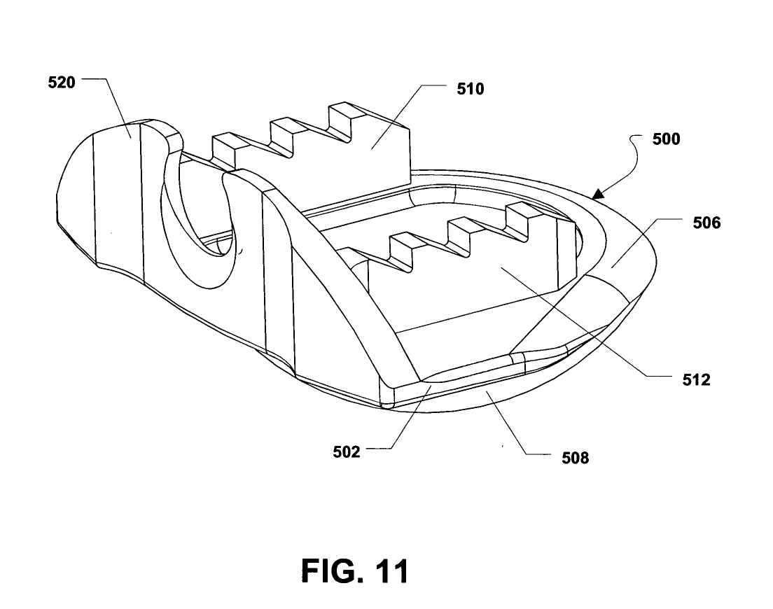

Referring to FIG. 9 through FIG. 12, details concerning the superior component 500 are illustrated. As shown, in a particular embodiment, the superior component 500 can include a superior support plate 502. The superior support plate 502 can include a superior articular surface 504 and a superior bearing surface 506. In a particular embodiment, the superior articular surface 504 and the superior bearing surface 506 can be substantially flat. In an alternative embodiment, the superior articular surface 504, the superior bearing surface, or a combination thereof can be at least partially curved.

As illustrated in FIG. 9 through FIG. 12, a projection 508 can extend from the superior articular surface 504 of the superior support plate 502. In a particular embodiment, the projection 508 can have a hemi-spherical shape. Alternatively, the projection 508 can have an elliptical shape, a cylindrical shape, or other arcuate shape.

FIG. 9 through FIG. 12 also indicate that the superior component 500 can include a first superior keel 510 and a second superior keel 512 that can extend from the superior bearing surface 506. Each superior keel 510, 512 can be configured to engage cortical bone, cancellous bone, or a combination thereof, after the intervertebral prosthetic disc 400 is installed within an intervertebral space between a superior vertebra and an inferior vertebra.

In a particular embodiment, after installation, the superior bearing surface 506 and the superior keels 510, 512 can be in direct contact with vertebral bone, e.g., cortical bone and cancellous bone. Further, the superior bearing surface 506 can be coated with a bone-growth promoting substance, e.g., a hydroxyapatite coating formed of calcium phosphate. Additionally, the superior bearing surface 506 and the superior keels 510, 512 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

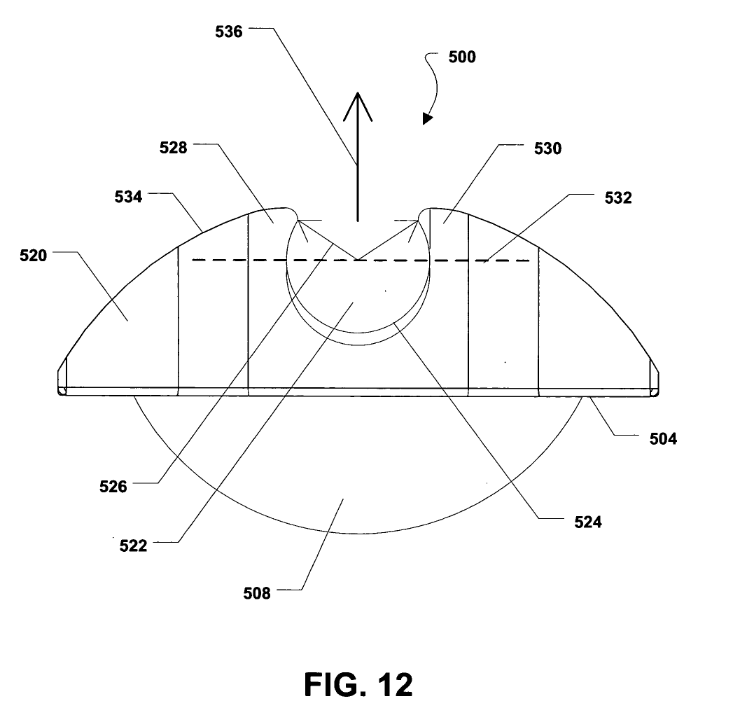

FIG. 9 through FIG. 11 show that the superior component 500 can include a superior bracket 520. In a particular embodiment, the superior bracket 520 can extend substantially perpendicular from the superior support plate 502. The superior bracket 520 can include a screw engagement hole 522 that is configured to engage the bone screw 700. In a particular embodiment, as shown in FIG. 12, the screw engagement hole 522 can be a semi-cylindrical hole having an inner surface 524. Further, the inner surface 524 of the screw engagement hole 522 can have a central angle 526 that can be greater than one hundred and eighty degrees (180°) and less than three hundred and sixty degrees (360°).

For example, the central angle 526 of the inner surface 524 of the screw engagement hole 522 can be two hundred degrees (200°), two hundred and ten degrees (210°), two hundred and twenty degrees (220°), two hundred and thirty degrees (230°), two hundred and forty degrees (240°), two hundred and fifty degrees (250°), two hundred and sixty degrees (260°), two hundred and seventy degrees (270°), two hundred and eighty degrees (280°), two hundred and ninety degrees (290°), three hundred degrees (300°), three hundred and ten degrees (310°), three hundred and twenty degrees (320°), three hundred and thirty degrees (330°), three hundred and forty degrees (340°), three hundred and fifty degrees (350°), or any other angle between one hundred and eighty degrees (180°) and three hundred and sixty degrees (360°).

Regardless of the central angle, the screw engagement hole 522 can be flanked by a first screw engagement structure 528 and a second screw engagement structure 530. In a particular embodiment, each screw engagement structure 528, 530 can be a portion of the material surrounding the screw engagement hole 522 between a central axis 532 of the screw engagement 522 and an upper perimeter 534 of the superior bracket 520. Accordingly, the screw engagement portions 528, 530 of the screw engagement hole 522 can partially wrap around the bone screw 700 (not shown in FIG. 12), and can substantially prevent the bone screw 700 from withdrawing from the screw engagement hole 522 in a radial direction, indicated by arrow 536. In certain embodiments, the screw engagement hole 522 can partially wrap around a head portion of the bone screw 700 or around a circumference of the head portion of the bone screw 700.

Referring back to FIG. 9, the bone screw 700 can be held in place within the superior bracket 520, e.g., within the screw engagement hole (not shown in FIG. 9) of the superior bracket 520, such that a longitudinal axis 702 of the bone screw 700 can be at an angle 704 with respect to the superior support plate 502. In a particular embodiment, the angle 704 can be in a range of five degrees to fifteen degrees (5°-15°). Further, the angle 704 can be in a range of seven degrees to thirteen degrees (7°-13°). Also, in a particular embodiment, the angle 704 can be in a range of nine degrees to eleven degrees (9°-11°).

### Description of the Inferior Component

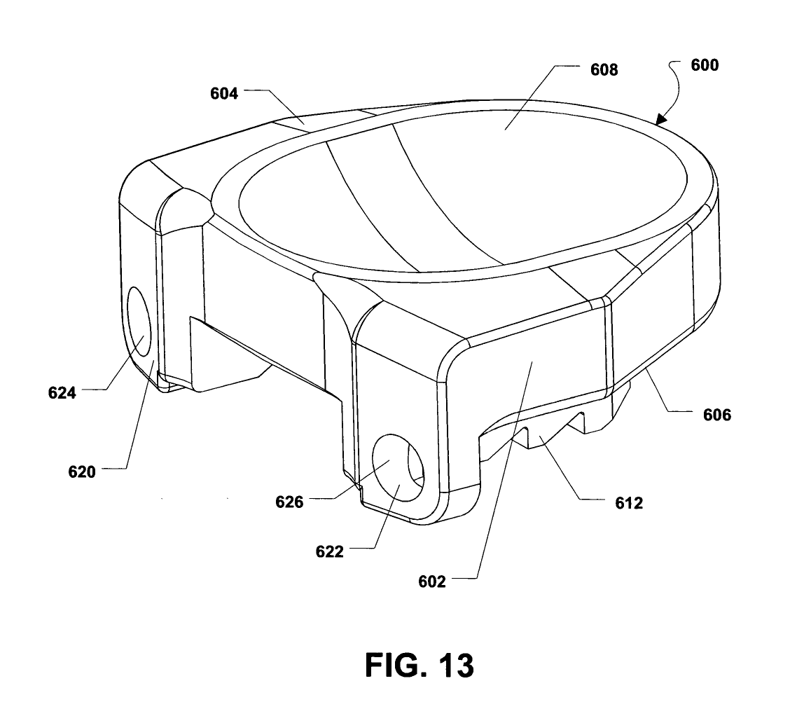

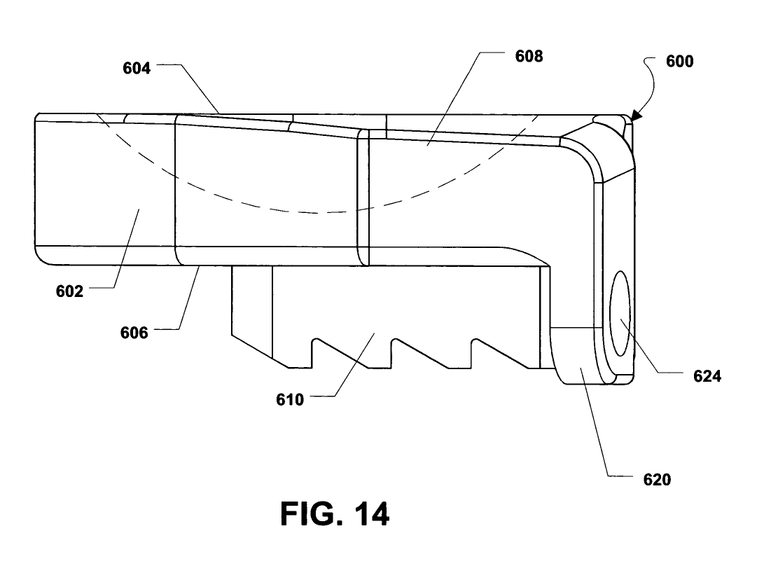

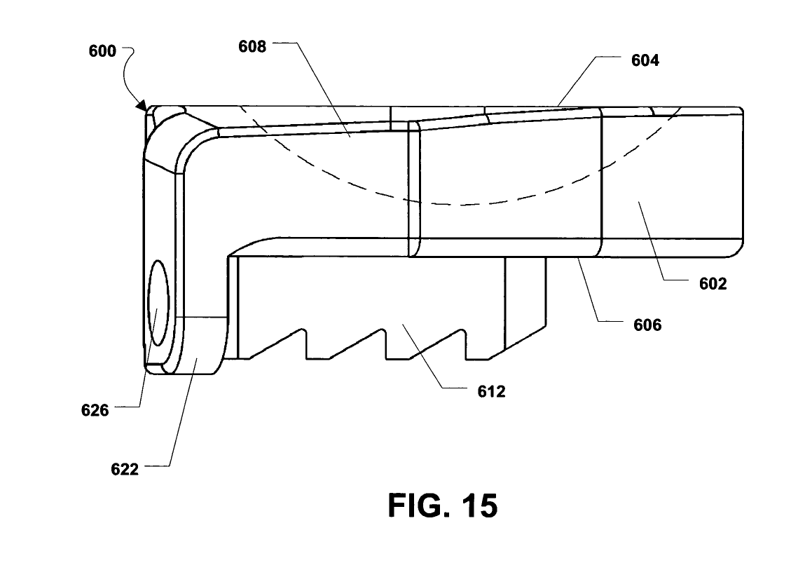

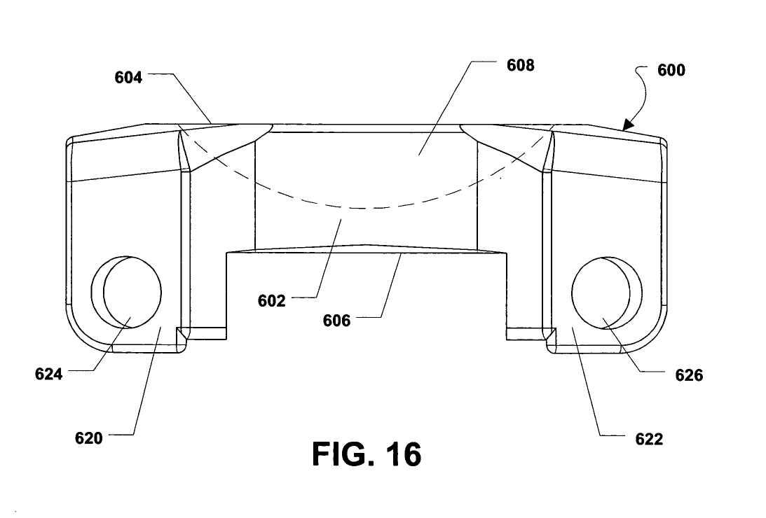

Referring to FIG. 13 through FIG. 16, details concerning the inferior component 600 are illustrated. As shown, in a particular embodiment, the inferior component 600 can include an inferior support plate 602. The inferior support plate 602 can include an inferior articular surface 604 and an inferior bearing surface 606. In a particular embodiment, the inferior articular surface 604 and the inferior bearing surface 606 can be substantially flat. In an alternative embodiment, the inferior articular surface 604, the inferior bearing surface, or a combination thereof can be at least partially curved.

As illustrated in FIG. 13 through FIG. 16, a depression 608 can extend into the inferior articular surface 604 of the inferior support plate 602. In a particular embodiment, the depression 608 can be sized and shaped to receive the projection 608 of the inferior component 600. For example, the depression 608 can have a hemi-spherical shape. Alternatively, the depression 608 can have an elliptical shape, a cylindrical shape, or other arcuate shape.

FIG. 13 through FIG. 16 also indicate that the inferior component 600 can include a first inferior keel 610 and a second inferior keel 612 that can extend from the inferior bearing surface 606. Each inferior keel 610, 612 can be configured to engage cortical bone, cancellous bone, or a combination thereof, after the intervertebral prosthetic disc 400 is installed within an intervertebral space between a inferior vertebra and an inferior vertebra.

In a particular embodiment, after installation, the inferior bearing surface 606 and the inferior keels 610, 612 can be in direct contact with vertebral bone, e.g., cortical bone and cancellous bone. Further, the inferior bearing surface 606 can be coated with a bone-growth promoting substance, e.g., a hydroxyapatite coating formed of calcium phosphate. Additionally, the inferior bearing surface 606 and the inferior keels 610, 612 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

FIG. 13 through FIG. 16 show that the inferior component 600 can include a first inferior bracket 620 and a second inferior bracket 622. In a particular embodiment, the inferior brackets 620, 622 can extend substantially perpendicular from the inferior support plate 602. The first inferior bracket 620 can include a first hole 624 established therethrough. Also, the second inferior bracket 622 can include a second hole 626 established therethrough.

Although depicted in the Figures as a two piece-design, in alternative embodiments, multiple-piece designs can be employed. For example, in an alternative embodiment, the projection 508 is not fixed or unitary with either of the support plates 502, 602 and, instead, is configured as a substantially rigid spherical member (not shown) that can independently articulate with each support plate 502, 602. Additionally or alternatively, each component can comprise multiple components (not shown). These components can articulate with or be fixed to the support plates 502, 602.

### Description of an Installation of the Intervertebral Prosthetic Disc within an Intervertebral Space

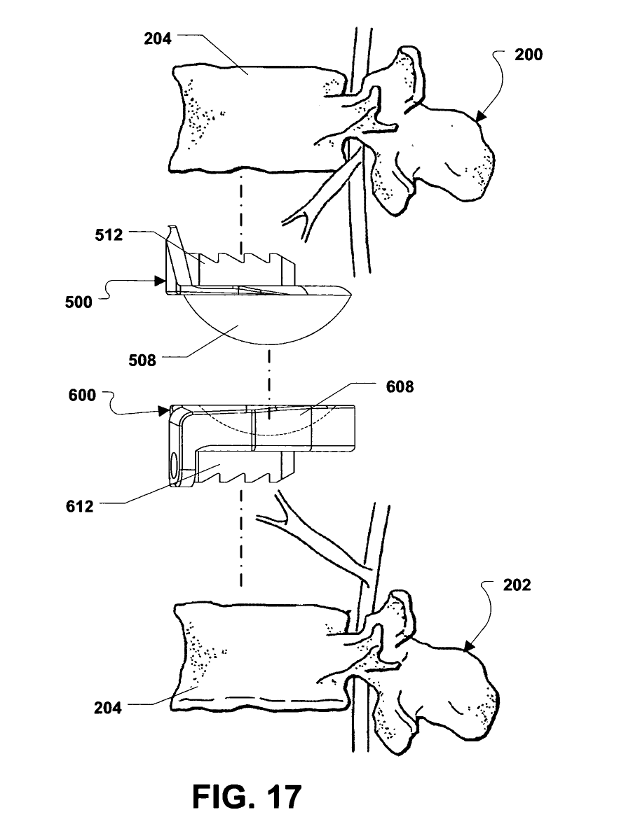

Referring to FIG. 17, an intervertebral prosthetic disc is shown between the superior vertebra 200 and the inferior vertebra 202, previously introduced and described in conjunction with FIG. 2. In a particular embodiment, the intervertebral prosthetic disc is the intervertebral prosthetic disc 400 described in conjunction with FIG. 4 through FIG. 16.

As shown in FIG. 17, the intervertebral prosthetic disc 400 can be installed within an intervertebral space 214 established between the superior vertebra 200 and the inferior vertebra 202 by removing vertebral disc material (not shown). In a particular embodiment, the superior keels 510, 512 of the superior component 500 can at least partially engage cortical bone, cancellous bone, or a combination thereof, of the superior vertebra 200. Further, the inferior keels 610, 612 of the superior component 600 can at least partially engage cortical bone, cancellous bone, or a combination thereof, of the inferior vertebra 202.

Also, in a particular embodiment, the projection 508 that extends from the superior component 500 of the intervertebral prosthetic disc 400 can at least partially engage the depression 608 that is formed within the inferior component 600 of the intervertebral prosthetic disc 400. It is to be appreciated that when the intervertebral prosthetic disc 400 is installed between the superior vertebra 200 and the inferior vertebra 202, the intervertebral prosthetic disc 400 allows relative motion between the superior vertebra 200 and the inferior vertebra 202. Specifically, the configuration of the superior component 500 and the inferior component 600 allows the superior component 500 to rotate with respect to the inferior component 600. As such, the superior vertebra 200 can rotate with respect to the inferior vertebra 202.

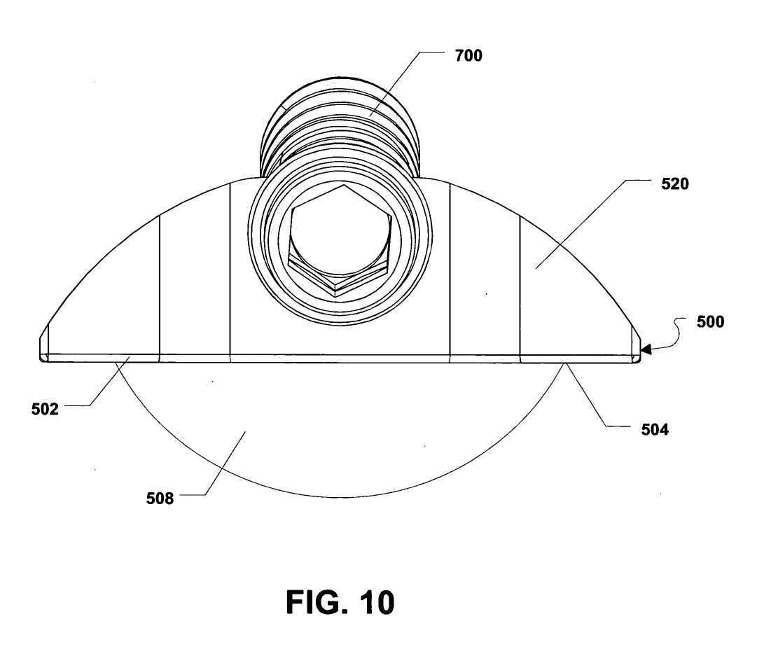

In a particular embodiment, the intervertebral prosthetic disc 400 can allow angular movement in any radial direction relative to the intervertebral prosthetic disc 400. Further, as depicted in FIG. 10 through 12, the inferior component 600 can be placed on the inferior vertebra 202 so that the center of rotation of the inferior component 600 is substantially aligned with the center of rotation of the inferior vertebra 202. Similarly, the superior component 500 can be placed relative to the superior vertebra 200 so that the center of rotation of the superior component 500 is substantially aligned with the center of rotation of the superior vertebra 200. Accordingly, when the vertebral disc, between the inferior vertebra 202 and the superior vertebra 200, is removed and replaced with the intervertebral prosthetic disc 400 the relative motion of the vertebrae 200, 202 provided by the vertebral disc is substantially replicated.

### Conclusion

With the configuration of structure described above, the intervertebral prosthetic disc provides a device that may be implanted to replace at least a portion of a natural intervertebral disc that is diseased, degenerated, or otherwise damaged. The intervertebral prosthetic disc can be disposed within an intervertebral space between an inferior vertebra and a superior vertebra. Further, after a patient fully recovers from a surgery to implant the intervertebral prosthetic disc, the intervertebral prosthetic disc can provide relative motion between the inferior vertebra and the superior vertebra that closely replicates the motion provided by a natural intervertebral disc. Accordingly, the intervertebral prosthetic disc provides an alternative to a fusion device that can be implanted within the intervertebral space between the inferior vertebra and the superior vertebra to fuse the inferior vertebra and the superior vertebra and prevent relative motion there between.

Further, the shape of the superior component, e.g., the shape of the screw engagement hole formed in the superior bracket of the superior component results in a relatively lower overall height of the intervertebral implant. In other words, since the screw engagement hole does not completely surround the bone screw, but extends around the shoulders of the bone screw, extra material that would otherwise increase the overall height of the intervertebral implant can be eliminated. The configuration of the screw engagement hole described herein can be incorporated in any implant device that can be fixed in place using one or more screws, e.g., an acetabular shell, a tibial base, a trauma plate, a cervical plate, etc.

In one or more alternative embodiments, the inferior component can be configured similarly to the superior component. In other words, the inferior component can include a bracket with an integrated bone screw. Further, the superior component, the inferior component, or a combination thereof can include a bracket with an integrated bone screw.

The above-disclosed subject matter is to be considered illustrative, and not restrictive, and the appended claims are intended to cover all such modifications, enhancements, and other embodiments that fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. An intervertebral prosthetic disc configured to be installed within an
intervertebral space between a superior vertebra and an inferior vertebra, the intervertebral prosthetic disc comprising:
an inferior component; and
a superior component movably engagable with the inferior component, wherein the superior component comprises:
a superior bracket; and
a screw engagement hole established in the superior bracket, wherein the screw engagement hole comprises a semi-cylindrical hole having an inner surface.

2. The intervertebral prosthetic disc of claim 1, wherein the screw engagement hole is configured to receive and engage a bone screw.

3. The intervertebral prosthetic disc of claim 1, wherein the inner surface of the screw engagement hole includes a central angle greater than one hundred and eighty degrees (180°) and less than three hundred and sixty degrees (360°).

4. The intervertebral prosthetic disc of claim 3, further comprising a first screw engagement structure and a second screw engagement structure adjacent to the screw engagement hole.

5. The intervertebral prosthetic disc of claim 4, wherein each screw engagement structure comprises a portion of the superior bracket between a central axis of the screw engagement hole and an upper perimeter of the superior bracket.

6. The intervertebral prosthetic disc of claim 5, further comprising a bone screw disposed within the screw engagement hole, wherein each screw engagement structure is configured to at least partially wrap around the bone screw disposed within the screw engagement hole and substantially prevent the screw from withdrawing from the screw engagement hole.

7. The intervertebral prosthetic disc of claim 1, wherein the superior component further comprises:
a superior support plate, wherein the superior bracket extends from the superior support plate; and
a projection extending from the superior support plate.

8. The intervertebral prosthetic disc of claim 7, wherein the inferior component further comprises:
an inferior support plate; and
a depression formed in the inferior support plate, wherein the depression is configured to receive the projection.

9. The intervertebral prosthetic disc of claim 8, wherein the inferior component further comprises:
a first keel extending from the inferior support plate; and
a second keel extending from the inferior support plate.

10. The intervertebral prosthetic disc of claim 9, wherein the inferior component further comprises:
a first inferior bracket extending from the inferior support plate;
a first hole formed in the first inferior bracket;
a second inferior bracket extending from the inferior support plate; and
a second hole formed in the second inferior bracket.

11. An intervertebral prosthetic disc configured to be installed within an
intervertebral space between a superior vertebra and an inferior vertebra, the intervertebral prosthetic disc comprising:
an inferior component having a depression formed therein; and
a superior component having a projection configured to movably engage the depression; and
a bone screw comprising a head portion integrated within the superior component, wherein the bone screw is held in place by a screw engagement hole that only partially surrounds the head portion of the bone screw.

12. The intervertebral prosthetic disc of claim 11, wherein the superior component comprises:
a superior support plate; and
a superior bracket extending from the superior support plate, wherein the screw engagement hole is formed in the superior bracket.

13. The intervertebral prosthetic disc of claim 12, wherein the screw engagement hole comprises a semi-cylindrical hole having an inner surface.

14. The intervertebral prosthetic disc of claim 13, wherein the inner surface of the screw engagement hole includes a central angle greater than one hundred and eighty degrees (180°) and less than three hundred and sixty degrees (360°).

15. The intervertebral prosthetic disc of claim 14, wherein the superior component further comprises:
a first screw engagement structure adjacent to the screw engagement hole; and
a second screw engagement structure adjacent to the screw engagement hole opposite the first screw engagement hole.

16. The intervertebral prosthetic disc of claim 15, wherein each screw engagement structure comprises a portion of the superior bracket between a central axis of the screw engagement hole and an upper perimeter of the superior bracket.

17. The intervertebral prosthetic disc of claim 16, wherein each screw engagement structure is configured to at least partially wrap around the bone screw disposed within the screw engagement hole and substantially prevent the screw from withdrawing from the screw engagement hole.

18. The intervertebral prosthetic disc of claim 12, wherein the bone screw is held in place within the superior bracket at an angle with respect to the superior support plate.

19. The intervertebral prosthetic disc of claim 18, wherein the angle is in a range of five degrees to fifteen degrees (5°-15°).

20. The intervertebral prosthetic disc of claim 19, wherein the angle is in a range of seven degrees to thirteen degrees (7°-13°).

21. The intervertebral prosthetic disc of claim 18, wherein the angle is in a range of nine degrees to eleven degrees (9°-11°).

22. An implant, comprising:
a component; and
a bone screw comprising a head portion integrated within the component, wherein the bone screw is held in place with respect to the component by a screw engagement hole that only partially surrounds the head portion of the bone screw.

23. The implant of claim 22, wherein the screw engagement hole comprises a semi-cylindrical hole having an inner surface.

24. The implant of claim 23, wherein the inner surface of the screw engagement hole includes a central angle greater than one hundred and eighty degrees (180°) and less than three hundred and sixty degrees (360°).

25. The implant of claim 24, wherein the component further comprises:
a first screw engagement structure adjacent to the screw engagement hole; and
a second screw engagement structure adjacent to the screw engagement hole opposite the first screw engagement hole.

26. The implant of claim 25, wherein each screw engagement structure comprises a portion of the superior bracket between a central axis of the screw engagement hole and an upper perimeter of the superior bracket.

27. The implant of claim 26, wherein each screw engagement structure is configured to at least partially wrap around the bone screw disposed within the screw engagement hole and substantially prevent the screw from withdrawing from the screw engagement hole.
